(19) United States
(12) Patent Application Publication
(10) Pub. No.: US 2022/0012634 A1
(43) Pub. Date: Jan. 13, 2022

(54) METHOD FOR ASSESSING DEGREE OF RISK OF SUBJECT AND CLASSIFYING SAME SUBJECT ACCORDING TO SAME DEGREE OF RISK, AND DEVICE USING SAME

(71) Applicant: VUNO INC., Seoul (KR)

(72) Inventor: Yeongnam LEE, Bucheon-si, Gyeonggi-do (KR)

(73) Assignee: VUNO INC., Seoul (KR)

(21) Appl. No.: 17/295,880

(22) PCT Filed: Dec. 4, 2019
(86) PCT No.: PCT/KR2019/017048

§ 371 Date: May 21, 2021

(30) Foreign Application Priority Data
Dec. 5, 2018 (KR) 10-2018-0154848

(51) Int. Cl. G06N 20/00; G16H 50/20; G16H 50/70; G06K 9/62

(52) U.S. Cl. CPC G06N 20/00; G16H 50/20; G06K 9/628; G06K 9/6256; G06K 9/6262; G16H 50/70

(57) ABSTRACT

The present disclosure provides a method for assessing the degree of risk of a subject and classifying the subject according to the degree of risk, and a computing device using same. Specifically, by a method according to the present invention, a computer device: obtains integrated data of the subject, wherein the integrated data, which is patient data relating to the subject or data obtained by processing the patient data, is numerical data; then applies the integrated data to a machine learning model for assessing the degree of risk of the subject to produce a result obtained by the classification, as a result obtained by assessing the degree of risk; and provides the produced classification result to an external entity.

### FIELD

The present disclosure relates to a method of assessing a criticality of a subject and classifying the subject based on the criticality and a computing device using the same. Specifically, according to the method according to the present disclosure, the computing device acquires integrated data of the subject, wherein the integrated data is patient data of the subject or data obtained by processing the patient data, and is numerical data, and thereafter, applies the integrated data to a machine learning model for criticality assessment of the subject to generate a result of the classification as a result of assessing the criticality, and then provides the generated result of the classification to an external entity.

### DESCRIPTION OF RELATED ART

In medical clinical trials, fatal symptoms of patients as subjects such as cardiac arrest and sepsis are dangerous symptoms with a very low survival and discharge-from-hospital percentage after their occurrence. For example, in the cardiac arrest, the survival and discharge-from-hospital percentage is only 20 to 30%. Since such cardiac arrest has a change in body data, in particular, bio-signal (vital signs) before its occurrence, early prediction thereof is possible at a certain degree. However, there is a problem in that accuracy of a conventional prediction method is low. This is mainly because the prediction depends on the experience or knowledge of the medical professional such as the nurse or doctor in charge. The subject or patient's criticality tends to be assessed quite differently depending on the competence of the individual. Further, there is a lack of the medical resources for such assessment itself, for example, manpower in the emergency department.

Therefore, it is necessary to efficiently manage the increasing emergency medical demand with limited medical resources, while accurately assessing patient's criticality. For example, the mortality of patients re-transferred after arriving at the emergency room is four times higher than that of patients as not re-transferred. As the reason for the re-transfer, the emergency room limit accounts for about 40%, and the shortage of medical staff accounts for about 32%.

Conventionally, in Korea, a method called “Korean Triage and Acuity Scale” has been used to solve this problem. However, there are three problems in this method. In other words, the real accuracy is low and subjective determination is required, such that there is a problem in that there is a difference in the results between the medical staffs and the decision-making itself for this purpose is not fast.

Therefore, the present disclosure proposes a criticality-based subject classification method that enables high-critical patients to receive treatment preferentially by more accurately and quickly classifying emergency room patients.

Prior technical literatures include Patent Document 1 KR10-1841222 B.

### DISCLOSURE

### Technical Purposes

A purpose of the present disclosure is to prioritize appropriate medical treatments or select a hospital to which the patient may transfer, based on the criticality that may lead to the subject's fatal symptoms, etc. in a medical site such as an emergency room or before arrival at the medical site.

Specifically, a purpose of the present disclosure is to quickly classifying patients using objective data.

### Technical Solutions

Characteristic configurations of the present disclosure to achieve the purposes of the present disclosure as described above and to realize the characteristic effect of the present disclosure to be described later are as follows.

According to one aspect of the present disclosure, there is provided a method for assessing a criticality of a subject and classifying the subject based on the criticality, the method comprising: (a) acquiring, by a computing device, integrated data of the subject or supporting, by the computing device, another device associated with the computing device to acquire the integrated data of the subject, wherein the integrated data is patient data of the subject or data obtained by processing the patent data, and is numericalized data; (b) applying, by the computing device, the integrated data to a machine learning model for criticality assessment of the subject to generate a result of the classification as a result of assessing the criticality, or supporting, by the computing device, said another device to apply the integrated data to the machine learning model to generate a result of the classification as a result of assessing the criticality; and (c) providing, by the computing device, the generated classification result to an external entity or supporting, by the computing device, said another device to provide the generated classification result to the external entity.

Preferably, the method further comprises (d) updating, by the computing device, the machine learning model, based on information obtained by assessing the classification result, or supporting, by the computing device, said another device to update the machine learning model, based on information obtained by assessing the classification result.

According to another aspect of the present disclosure, there is provided a computer program comprising instructions stored on a medium, wherein the instructions are implemented to cause a computing device to perform the method according to the present disclosure.

According to still another aspect of the present disclosure, there is provided a computing device configured to assess a criticality of a subject and classify the subject based on the criticality, the device comprising: a communication unit configured to acquire integrated data of the subject; and a processor configured to perform: (i) a process of applying the integrated data to a machine learning model for criticality assessment of the subject to generate a result of the classification as a result of assessing the criticality, or of supporting another device connected to the computing device via the communication unit to apply the integrated data to the machine learning model to generate a result of the classification as a result of assessing the criticality; and (ii) a process of providing the generated classification result to an external entity or of supporting said another device to provide the generated classification result to the external entity, wherein the integrated data is patient data of the subject or data obtained by processing the patent data, and is numericalized data.

Preferably, the processor of the computing device further performs a process of updating the machine learning model, based on information obtained by assessing the classification result, or of supporting said another device to update the machine learning model, based on information obtained by assessing the classification result.

### Technical Effects

According to an exemplary embodiment of the present disclosure, the patient may be classified according to the possibility of the occurrence of specific symptoms such as fatal symptoms more rapidly, compared to the conventional scheme of determining the patient's criticality and classifying the patient, based on the experience or knowledge of the medical experts. In other words, the present disclosure has the effect of determining the patient's criticality in the medical field and setting the priority of the treatment based on the criticality or recommending a ward or hospital that is suitable for the patient's criticality.

As a result, the medical staff may save the time, and the safety for more patients may be secured.

Further, according to the exemplary embodiment, the patient data recorded in the conventional medical field may be used as it is, thereby innovating a workflow in the medical field without building an additional system.

### BRIEF DESCRIPTION OF THE DRAWINGS

The accompanying drawings which are attached to be used to describe the embodiments of the present disclosure are only some examples of the present disclosure. Other drawings may be obtained based on these drawings by a person with ordinary knowledge in the technical field to which the present disclosure belongs (hereinafter, referred to as “skilled person to the art”) without any inventive work.

FIG. 1 is a diagram showing a main concept to describe a recurrent neural network (RNN) as an example of a machine learning model used in an embodiment of the present disclosure.

FIG. 2 is a conceptual diagram schematically showing an example configuration of a computing device that performs a method (hereinafter referred to as “criticality-based subject classification method”) for assessing a criticality of a subject and classifying the subject based on the criticality thereof according to an embodiment of the present disclosure.

FIG. 3 is a conceptual diagram illustrating an example hardware and software architecture of a computing device performing a criticality-based subject classification method according to an embodiment of the present disclosure.

FIG. 4 is a first example diagram for illustrating integration of patient data as performed in an embodiment of the present disclosure.

FIG. 5 is a second example diagram for illustrating integration of patient data performed in an embodiment of the present disclosure.

FIG. 6 is a conceptual diagram to illustrate a method of generating similar specific symptom occurrence data using a generative adversarial network (GAN) in an embodiment of the present disclosure.

FIG. 7 is a flowchart illustrating steps of a criticality-based subject classification method according to an embodiment of the present disclosure.

### DETAILED DESCRIPTIONS

The detailed descriptions of the present disclosure to be described later refer to the accompanying drawings which illustrate specific embodiments in which the present disclosure may be implemented by way of example in order to clearly present the objectives, technical solutions, and advantages of the present disclosure. These embodiments will be described in a sufficiently detailed manner to enable a person skilled in the art to implement the present disclosure. In the description with reference to the accompanying drawings, the same reference numerals are assigned to the same elements regardless of the reference numerals, and redundant descriptions thereof will be omitted.

Specific structural or functional descriptions of the embodiments may be disclosed for purposes of illustrations only, and may be changed and implemented into various forms. Accordingly, the embodiments are not limited to specific forms as disclosed, and the scope of the present specification includes changes, equivalents, or substitutes included in the technical idea.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

It will be understood that when an element or layer is referred to as being “connected to”, or “coupled to” another element or layer, it may be directly on, connected to, or coupled to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Further, throughout the detailed description and claims of the present disclosure, ‘learning’ is a term that refers to performing machine learning via computing according to a procedure. Thus, it will be appreciated by those of ordinary skill in the art that this term is not intended to refer to mental processes such as human educational activities.

Further, in the detailed description and claims of the present disclosure, the term ‘patient data’ and variations thereof refer to body data, that is, text obtained by assessing the user's body, or various types of data measured from the user's body. It should be understood that the patient data is a concept including not only at least one of bio-signal and body composition data, but also various non-body data related to patient treatment, e.g., a type and a location of a medical institution, locations of other adjacent medical institutions or a distance to the patient. It will be appreciated by those skilled in the art that such patient data may be measured based on, for example, contact with the user's body, but is not limited thereto. For example, the patient data that may include body weight, body fat percentage, skeletal muscle mass, body fat mass, body water content, fat-free mass (FFM), FFMI (FFM/height²), and SMMI (skeletal muscle mass/height²), BFMI (body fat mass/height²), BMI (weight/height²), ASM (leg and arm muscle mass), PBF (body fat mass/body weight), and blood pressure.

Further, it should be understood that throughout the detailed descriptions and claims of the present disclosure, ‘bio-signal’ is not meant to be construed as being limited only to its usual meaning referring to measurements of a body temperature, electrocardiogram, respiration, pulse, blood pressure, oxygen saturation, skin conductivity, etc. of the subject, but includes the amount and concentration of specific substances in biological samples that may be obtained through EEG signals and other measurements.

In this connection, the ‘biological sample’ should be understood as various kinds of substances that may be collected from the subject such as blood, serum, urine, lymph, cerebrospinal fluid, saliva, semen, vaginal fluid, etc. of the subject.

Further, it should be understood that throughout the detailed descriptions and claims of the present disclosure, the word ‘fatal symptom’ and its variations are not limited to cardiac arrest as an example of an object to which the present disclosure applies, and are concepts including various kinds of clinical phenomena that may cause a great risk to the life of the subject based on time series changes such as sepsis. In addition, the word ‘specific symptoms’ and its variations are terms that refer to various symptoms that may be significantly identified in clinical practice, including fatal symptoms.

Moreover, the present disclosure covers all possible combinations of the embodiments presented herein. It should be understood that the various embodiments of the present disclosure are different from each other, but need not be mutually exclusive. For example, specific shapes, structures, and characteristics as described herein in relation to an embodiment may be implemented in other embodiments without departing from the spirit and scope of the present disclosure. Further, it should be understood that the location or arrangement of individual components in each of the disclosed embodiments may be changed without departing from the spirit and scope of the present disclosure. Accordingly, the detailed description to be described below is not intended to be taken in a limiting sense, and the scope of the present disclosure, if properly described, is limited only by the appended claims, along with all scopes equivalent to those claimed by the claims. Similar reference numerals in the drawings refer to the same or similar functions in terms of several aspects.

Unless otherwise indicated or clearly contradicted by context in this specification, items referred to in the singular form include the plural forms thereof. Further, in describing the present disclosure, when it is determined that a detailed description of a related known component or function may obscure the gist of the present disclosure, the detailed description thereof will be omitted.

Hereinafter, preferred embodiments of the present disclosure will be described in detail with reference to the accompanying drawings in order to facilitate the implementation of the present disclosure.

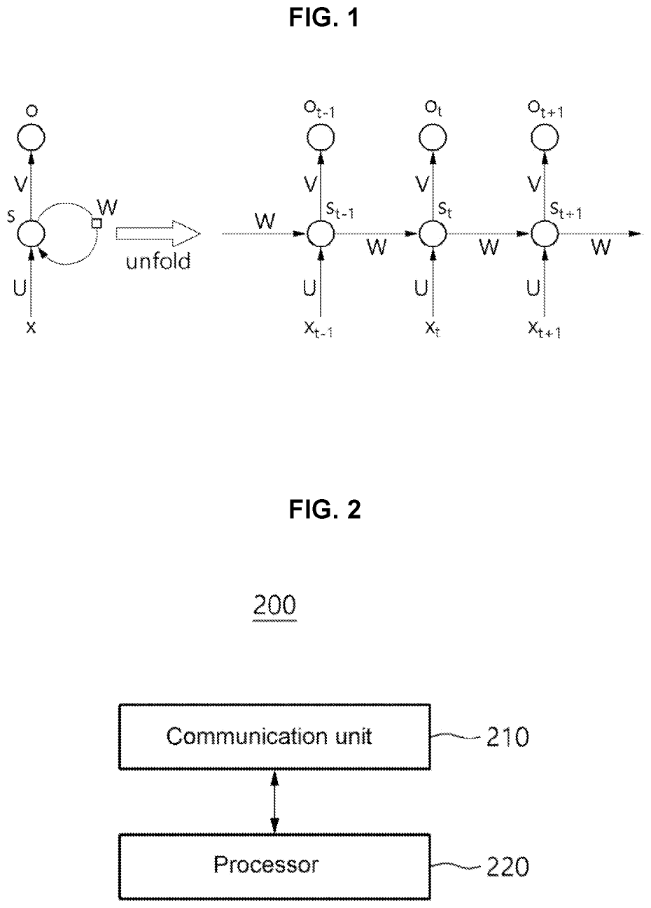

FIG. 1 is a diagram showing the main concept to describe a recurrent neural network as an example of a machine learning model used in an embodiment of the present disclosure.

Referring to FIG. 1, the deep neural network model among the machine learning models used in the present disclosure may be briefly described in a form of stacking artificial neural networks in multiple layers. In other words, the deep neural network model is expressed as a deep neural network or a deep neural network in the sense of a deep-structured network. The deep neural network model is a machine learning model that learns a large amount of data to be analyzed in a structure of a multi-layered network, and automatically learns the characteristics of each data to be analyzed and the relationship between the data to be analyzed, and thus performs learning in a way that minimizes the error of the prediction result of the target function, that is, a specific symptom.

A recurrent neural network (RNN) as one example of a deep neural network model used in the present disclosure may be used to analyze sequentially input time-series data, as shown in FIG. 1. This deep neural network is structured to find features of data according to a time order, and select and reflect a main feature to be referred to in analysis at a current time-point among features of a previous time-point. For example, referring to FIG. 1, when this deep neural network analyzes data input at a t+1 time-point, the network may analyze the data via the learning reflecting the main features analyzed at a t−1 time-point and a t time-point. As described above, in accordance with the present disclosure, changes in the data over time may be extracted using the structure of the recurrent neural network and may be utilized to assess the criticality of a patient through prediction of specific symptoms.

In short, the recurrent neural network that develops along a time-series sequence, a time flow, or a time axis may be understood as a deep neural network with infinite layers. Thus, referring to FIG. 1, xₜ refers to the input vector at time-point t, and sₜ refers to the hidden state (i.e., memory of a neural network) at the time-point t.

In addition, the recurrent neural network conceptually shown in FIG. 1 follows the equations of sₜ=f(U xₜ+W sₜ₋₁) and y=g(V sₜ). The generalized neural network follows the equations sₜ=f(xₜ, sₜ₋₁, Uᵢ, . . . ), y=g(sₜ, Uⱼ, . . . ), U=U₁, W=U₂, V=U₃, and i may be 3 or greater.

For reference, y is denoted by o in FIG. 1. In this connection, f refers to an activation function (e.g., tanh( )and ReLU function), and U, V, and W refer to parameters of a neural network. In this regard, U, V, and W are parameters that are equally shared across all time-point steps in a recurrent neural network, unlike in a feedforward neural network. g is the activation function (typically, there is a softmax function) for an output layer, and y is the output vector of the neural network at the t time-point. An embodiment of the present disclosure using such a recurrent neural network will be described in detail later.

Next, FIG. 2 is a conceptual diagram schematically showing an example configuration of a computing device that performs a criticality-based subject classification method according to an embodiment of the present disclosure.

Referring to FIG. 2, a computing device 200 according to an embodiment of the present disclosure includes a communication unit 210 and a processor 220, and may communicate directly or indirectly with an external computing device (not shown) via the communication unit 210.

Specifically, the computing device 200 may achieve desired system performance using a combination of typical computer hardware (e.g., a computer, a processor, a memory, a storage, an input device and an output device, and other components of a conventional computing device; electronic communication devices such as routers and switches; electronic information storage systems such as network-attached storage (NAS) and storage area network (SAN)) and computer software (i.e., instructions that cause the computing device to function in a specific way). The storage may include not only a storage device such as a hard disk or a universal serial bus (USB) memory, but also a storage device based on a network connection such as a cloud server.

The communication unit 210 of such a computing device may communicate requests and responses with other computing devices that are associated therewith. In an example, such a request and a response may be made by the same transmission control protocol (TCP) session, but are not limited thereto. Alternatively, such a request and a response may be transmitted and received as, for example, a user datagram protocol (UDP) datagram.

Specifically, the communication unit 210 may be implemented in the form of a communication module including a communication interface. For example, the communication interfaces may include wireless Internet interfaces such as WLAN (wireless LAN), WiFi (wireless fidelity) Direct, DLNA (digital living network alliance), Wibro (wireless broadband), Wimax (world interoperability for microwave access), HSDPA (high speed downlink packet access), etc. and a short-range communication interface such as Bluetooth™, radio frequency identification (RFID), infrared data association (IrDA), ultra-wideband (UWB), ZigBee, and near field communication (NFC). In addition, the communication interface may include all interfaces (e.g., wired interfaces) capable of performing communication with an external component.

For example, the communication unit 210 may acquire patient data including a user's bio-signal, body data such as blood test data, and non-body data from an external device through an appropriate communication interface. In addition, in a broad sense, the communication unit 210 may include or be associated with a keyboard, a mouse, other external input devices, printing devices, displays, and other external output devices for receiving instructions or commands.

Further, the processor 220 of the computing device may include hardware components such as a micro processing unit (MPU), a central processing unit (CPU), a graphics processing unit (GPU), a neural processing unit (NPU), or a tensor processing unit (TPU), and a cache memory), data bus, and the like. Further, the processor may further include an operating system and a software configuration of an application that performs a specific purpose.

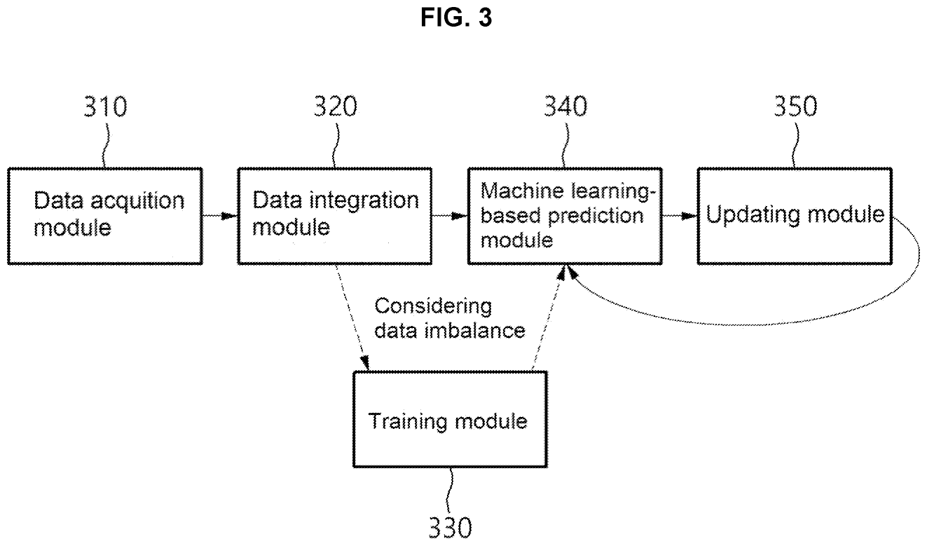

FIG. 3 is a conceptual diagram illustrating an example hardware and software architecture of a computing device performing a criticality-based subject classification method according to an embodiment of the present disclosure.

Referring to FIG. 3, in an overview of the method and device configuration according to the present disclosure, the computing device 200 may include a data acquisition module 310 as a component thereof. It is understood by those of ordinary skill in the art that the data acquisition module 310 may be implemented via the communication unit 210 included in the computing device 200 or via the association between the communication unit 210 and the processor 220.

The data acquisition module 310 may acquire patient data of a subject, for example, body data such as bio-signal, blood test data, and non-body data including various data related to subject or patient handling, such as the type and location of the related medical institution, locations of other adjacent medical institutions, or the distance thereof to the patient. For example, these data may be obtained from the subject's electronic medical record (EMR). However, the disclosure is not limited thereto.

Next, the patient data above may be transferred to a patient information integration module 320. The data integration module 320 generates numericalized integrated data by integrating numerical data included in the patient data with data that may be expressed in a text form. In this connection, the data that may be expressed in the text form may include text itself or voice data. Clinical notes and prescriptions may be included in the data that may be expressed in the text form. Further, for example, the bio-signal may be expressed in numerical terms, and chief complaints may be expressed in the textual data. Thus, the initial patient data is expressed in various forms. In order to easily use those data for machine learning, it is desirable to transform those data so that those data are expressed in the same form. This data integration will be described in detail as follows.

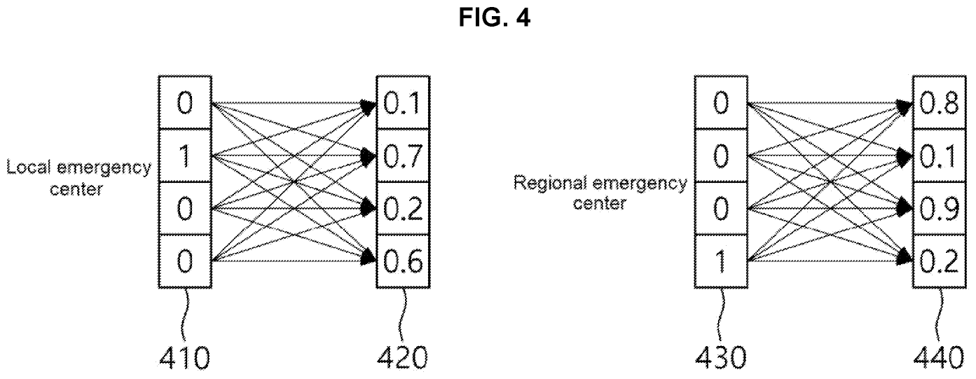

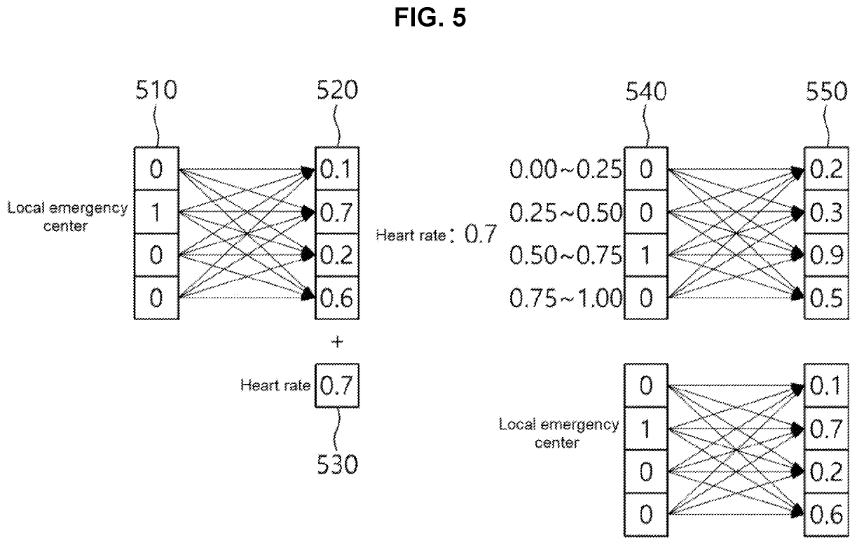

FIG. 4 is a first example diagram for illustration of the integration of patient data performed in an embodiment of the present disclosure, and FIG. 5 is a second example diagram for illustration of the integration of patient data.

Referring to FIG. 4, an example in which text data is converted to numerical data is shown. Information about a regional emergency center which is an example of text data may be first expressed as categorized data 410, and this categorized data 410 may be expressed in numerical form 420. For example, for a method for processing text data into categorized data 410 and then the categorized data 410 into the numerical form 420, a word embedding technique, for example, word2vec technique may be used.

In FIG. 4, another example of text data, that is, the information about the regional emergency center may be expressed in categorized data 430, and the categorized data 430 may be processed into numerical form 440. In another example, since the text data may include not only one word but also several words, in that case, the categorization and numerical processing may be performed on each word included in the text data.

However, the data in which the text is expressed in the numerical form has more influence on training than the original numerical data in the form of the numerical data has. This may lead to a problem that the data are not evenly reflected in the machine learning. This may be because, for example, as shown in FIG. 5, while the regional emergency center is expressed based on data 520 of a 4×1 matrix through categorization 510, while the heart rate 530 may be expressed based on a 1×1 matrix.

As a way to solve this problem, the present inventors allow the numerical data to be converted to a same form as the text is categorized and then expressed in the numerical form. That is, referring to FIG. 5, the heart rate may be categorized into a class of 0.00 to 0.25, a class of 0.25 to 0.50, a class of 0.50 to 0.75, and a class of 0.75 to 1.00. In this connection, the heart rate 0.7 may be categorized and expressed in (0,0,1,0)^{T} 540. This categorized data may be converted back to the numeric form 550. Thus, one scheme of such conversion may include word embedding as described above. For reference, the heart rate shown in FIG. 5 is scaled (or normalized) to have a value of 0 to 1.0. This value may be adapted as input/output values to/from machine learning, especially, artificial neural networks. In this way, the numerical data may be scaled to have a value of 0 to 1.0.

Numerical data that is expressed in the numerical form through the categorization as described above may have the same form as data in which the text is expressed in numerical terms. Thus, this may be referred to as so-called integration.

In other words, the integrated data in the numerical form is patient data of the subject or data obtained by processing the patient data, and is numericalized data and in turn is transmitted to a machine learning-based prediction module 340 for criticality assessment of the subject. Then, the machine learning-based prediction module 340 functions to generate a classification result as a result of assessing the criticality of the subject. A specific process thereof will be described later.

In one example, a training module 330 for training the machine learning-based prediction module 340 may receive and use the provided integrated data as it is. In this case, most of the integrated data is normal such that the patient may be discharged from the emergency room. However, since the number of integrated data of patients with high criticality is small, that is, data imbalance is present, a situation occurs where the machine learning model is trained mainly based on the normal data.

In order to solve this problem, in accordance with the present disclosure, the training module 330 may more sufficiently consider the integrated data of a patient with high criticality, for example, a patient corresponding to the occurrence of a specific symptom such as cardiac arrest. Specifically, the training module 330 adjusts the sample ratio of the input integrated data, and initially learns only specific symptom occurrence data or mainly learns specific symptom occurrence data and then learns the specific symptom occurrence data and normal data simultaneously at the same or similar ratio.

However, since in the medical reality, the number of specific symptom occurrence data is significantly smaller than that of normal data that may be actually obtained, similar (or fake) specific symptom occurrence data similar to the real one may be generated as a substitute for the specific symptom occurrence data and may be used for the simultaneous learning. Means for obtaining the data similar to the real data may include various means widely known to those skilled in the art, including generative adversarial networks, variable autoencoders, and the like. For example, the specific configuration of the generative adversarial network is disclosed in “Generative Adversarial Networks” by Goodfellow, Ian J.; Pouget-Abadie, Jean; Mirza, Mehdi; Xu, Bing; Warde-Farley, David; Ozair, Sherjil; Courville, Aaron; Bengio, Yoshua (2014).

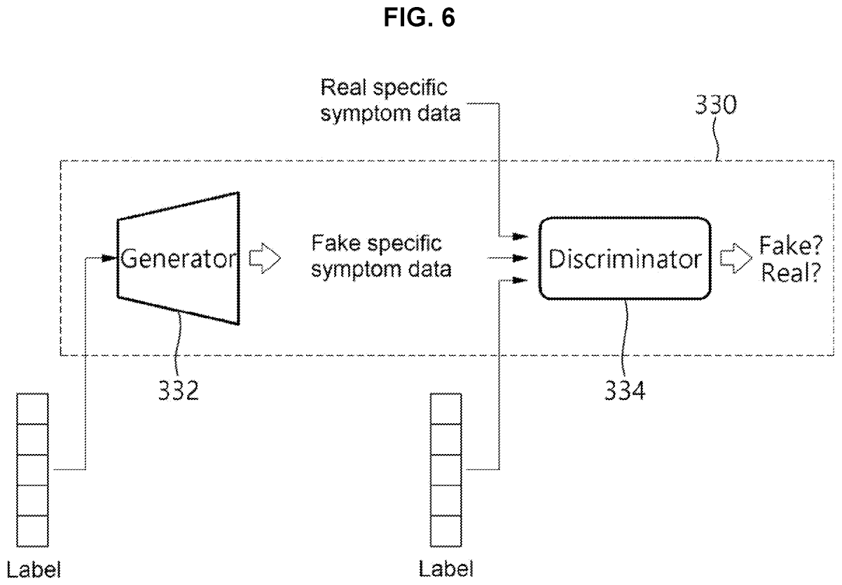

FIG. 6 is a conceptual diagram to illustrate a method for generating similar (or fake) specific symptom occurrence data using such a generative adversarial network (GAN) in an embodiment of the present disclosure.

Referring to FIG. 6, in this embodiment, the generative adversarial network (GAN) included in the training module 330 includes a generator 332 and a discriminator 334. The generator receives a predetermined label and generates similar (or fake) specific symptom occurrence data similar to real one based on this label to trick the discriminator to discriminate the similar (or fake) specific symptom occurrence data as real specific symptom occurrence data. The discriminator aims to distinguish between the real specific symptom occurrence data and the generated similar (or fake) specific symptom occurrence data. In the process of the learning using this GAN, the generator and the discriminator update the neural network weights to achieve respective goals. Thus, it turns out that after sufficient learning, the generator generates the similar (or fake) specific symptom occurrence data similar to the real one, and the discrimination ratio by the discriminator theoretically converges to 0.5.

As a result, the generator sufficiently trained using the GAN generates data (i.e., similar (or fake) specific symptom occurrence data) similar to the real specific symptom occurrence data. Thus, the desired sample ratio to solve the data imbalance and learn specific symptom occurrence data and normal data at the same time may be obtained. This is because the generator may sufficiently generate the similar (or fake) specific symptom occurrence data similar to the real one at a level equivalent to normal data. It will be understood by those of ordinary skill in the art that the above description is not intended to limit the present disclosure to that of using GAN.

Next, the updating module 350 pre-learns the machine learning-based prediction module 340 (or more precisely, the machine learning model adopted in the machine learning module) used to predict the specific symptoms of the subject or serves to update the machine learning model based on the information obtained by (medical staff, etc.) assessing the classification result according to the method of the present disclosure.

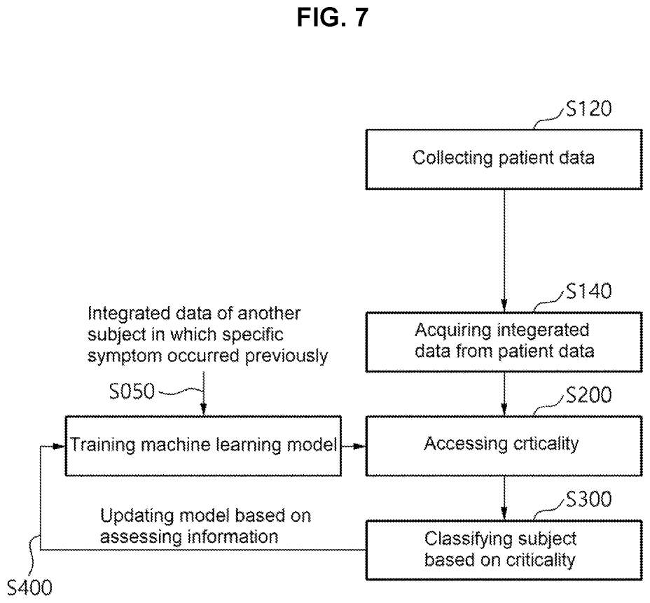

Now, the criticality-based subject classification method according to the present disclosure will be described in detail with reference to FIG. 7. FIG. 7 is a flowchart illustrating an example of a criticality-based subject classification method according to an embodiment of the present disclosure.

Referring to FIG. 7, in the criticality-based subject classification method according to the present disclosure, first, the data acquisition module 310 implemented (or executed) by the communication unit 210 of the computing device 200 may acquire the integrated data of the subject or support the other device linked to the computing device to acquire the integrated data thereof (S120, S140). The integrated data is the subject's patient data (S120) or data obtained by processing the subject's patient data and is numericalized data (S140).

In an embodiment of step S120, the data integration module 320 implemented by the processor 220 of the computing device 200 may generate categorized data by categorizing non-numerical data among the patient data and may convert the non-numerical data into the numerical data by processing the categorized data into numerical form. As described above with reference to FIG. 4, examples of the categorized data generated by categorizing non-numerical data are denoted by reference numerals 410 and 430 shown in FIG. 4. An example of the result of the categorized data being processed in the numerical form is denoted by reference numerals 420 and 440 shown in FIG. 4.

Preferably, in step S120 of this embodiment, the data integration module 320 performs the categorization and numerical processing of the numerical data among the patient data so as to correspond to the result of converting the non-numerical data into the numerical data. As described above with reference to FIG. 5, an example of the result of converting the non-numerical data (e.g., information about local emergency center) into the numerical data is denoted as the reference numeral 520 shown in FIG. 5. The result of performing the categorization of the numerical data (e.g., heart rate) among the patient data and the result of performing the numerical processing on the result are denoted as reference numerals 540 and 550 shown in FIG. 5, respectively.

Next, referring to FIG. 7, the criticality-based subject classification method further includes steps (S200, S300) in which the machine learning-based prediction module 340 implemented by the processor 220 of the computing device 200 applies the integrated data in steps S120 and S140 to a machine learning model for criticality assessment of the subject to generate the result of the classification as a result of assessing the criticality (S200) or supports the other device to generate the result of the classification as a result of assessing the criticality (S300).

In this connection, the subject's criticality may be classified into at least four classes, which may be, for example, a dischargeable patient, a hospitalization requested patient, an ICU treatment requested patient, and an impending death patient.

Specifically, the assessment of criticality (S200) may be performed via prediction of occurrence of specific symptoms, for example, cardiac arrest having the high criticality. To this end, the result obtained by the machine learning model predicting the occurrence of the specific symptom from the current time-point t of the integrated data to the time-point t+n time-point after a predetermined time interval n since the current time-point t may be used.

In a specific example, the machine learning model may include a deep neural network model such as a recurrent neural network model, which may be executed by the processor 220. In the recurrent neural network following the equations of sₜ=f(U xₜ+W sₜ₋₁) and y=g(V sₜ) (the generalized deep neural network thereof follows the equations sₜ=f(xₜ, sₜ₋₁, Uᵢ, . . . ), y=g(sₜ, Uⱼ, . . . ), U=U₁, W=U₂, V=U₃, and i may be 3 or greater) as described above, the xₜ refers to the integrated data, which is an input vector at t time-point, or a value processed from the integrated data. The value processed from the integrated data may be, for example, a change amount (from a previous time-point to a corresponding time-point) of the integrated data or a change in the change amount.

Further, the sₜ refers to a hidden state corresponding to the memory of the recurrent neural network model at the t time-point. The sₜ₋₁ refers to the hidden state at t−1 time-point. The U, V and W refer to neural network parameters that are shared equally over all time-points of the recurrent neural network model. The f refers to a first activation function selected to yield the hidden state. The y denotes an output layer as a latent feature according to the recurrent neural network model at time-point t, and the g denotes a second activation function selected to calculate the output layer.

Specifically, a first half portion (not shown) of a machine learning model according to the present disclosure serves to reflect the relationship between the integrated data, with referring to the integrated data at the t time-point, and thus may correspond, for example, to U xₜ in the recurrent neural network model. In addition, a second half portion (not shown) of the machine learning model according to the present disclosure reflects the change in the integrated data over time, with referring to the integrated data up to the t−1 time-point, and thus may correspond to, for example, W _{St-1} in the recurrent neural network model.

In this connection, the first activation function f may be a commonly used tanh( )or ReLU function. Further, the second activation function g may be a commonly used softmax function. It is known that the selection of the first activation function and the second activation function may vary depending on the use thereof, and depending on the complexity of the calculation thereof.

Further, in this example, the machine learning model may further include at least one fully connected layer for calculating the probability of occurrence of the specific symptom from the output layer y.

However, the machine learning model for assessing the criticality is not limited to deep neural networks of such recurrent neural networks. Thus, the skilled person to the art may utilize various neural network architectures suitable for criticality assessment.

Further, the machine learning model may be trained using occurrence data of specific symptoms, and normal data, as described above with respect to the machine learning-based prediction module 340.

In this case, preferably, the machine learning model may be trained first through (i) learning specific symptom occurrence data, and then (ii) learning specific symptom occurrence data and normal data at the same time. In addition, as described above, in the simultaneous learning step of (ii), data imbalance may be solved by generating and using similar (or fake) specific symptom occurrence data similar to the real one.

In this way, before performing the criticality-based subject classification method according to the present disclosure, the machine learning model needs to be pre-trained (S050). To this end, the training module 330 may be executed by the processor 220.

For the learning of the deep neural network (for example, recurrent neural network) model, the training module 330 may train the deep neural network model through back-propagation using a plurality of integrated data as training data. Through such training, parameters, weights, and bias values of the deep neural network may be determined. As mentioned above, the machine learning model that may be used in the present disclosure is not limited to deep neural networks such as recurrent neural networks. Various modifications widely known in the technical field of the present disclosure, such as long-short term memory (LSTM), GRU, and MemN, may be used.

Then, referring to FIG. 7, the criticality-based subject classification method according to the present disclosure further includes a step (not shown) in which in order to promote useful use of the result, a predetermined module (not shown) implemented by the computing device 200 provides the result of the classification to an external entity.

In this connection, it should be understood that the external entity includes the user and administrator of the computing device 200 performing the method according to the present disclosure and the medical professional in charge of the subject, and further includes any entity that requires a classification result. When the external entity is a human, the computing device 200 may provide the classification result to the external entity through a predetermined output device, for example, a user interface displayed on a display.

Until now, the components shown in FIG. 2 to FIG. 7 have been exemplified as being realized in one computing device for convenience of explanation. However, it will be appreciated that the computing device 200 performing the method according to the present disclosure may be embodied as a plurality of devices as connected to each other. Therefore, it is obvious that each step of the method according to the present disclosure as described above may be performed either by one computing device directly or by one computing device supporting the other computing device associated with the one computing device to perform each step.

In this way, the criticality-based subject classification method according to the present disclosure may assess the criticality of the subject and classify the subject based on the pre-trained machine learning model. When the information obtained by the user, etc. assessing the classification result is used as data for updating the machine learning model again, the machine learning model may perform more accurate classification. Thus, the criticality-based subject classification method according to the present disclosure may further include a step (S400) in which the processor 220 updates the machine learning model or supports the other device to update the model, based on the information obtained by assessing the classification result. In this connection, the accuracy of the machine learning model is improved because the integrated data which was not considered in the previous learning is additionally considered as the training data, such that errors in the previous learning may be corrected. Thus, the performance of the machine learning model continuously improves as data accumulates.

In this connection, the information obtained by assessing the classification result may be provided from an external entity such as the medical professional. For example, in order not to erroneously distinguish between a dischargeable patient, a hospitalization requested patient, a patient requiring thew ICU treatment, and an impending death patient, the external entity immediately corrects the error of the incorrectly classified classification based on the information obtained by assessing the classification result.

As described above, in accordance with all the above-described embodiments of the present disclosure, efficient coping and treatment according to rapid patient classification may be realized with only data measured or used in the emergency room even in environments where medical resources are limited, thereby quickly saving the life of a high-risk patient, and reducing the effort of medical staff for patient classification.

Based on the description of the various embodiments of the present disclosure, a person skilled in the art may clearly understood that the method and/or processes according to the present disclosure, and the steps thereof may be realized by hardware, software, or any combination of the hardware and software suitable for a particular application. The hardware may include a general purpose computer and/or a dedicated computing device or a specific computing device or a special feature or component of a specific computing device. The processes may be realized by one or more processors with internal and/or external memory, for example, microprocessors, controllers, e.g. microcontrollers, embedded microcontrollers, microcomputers, arithmetic logic units (ALUs), digital signal processors, for example, a programmable digital signal processor or other programmable device. In addition, or as an alternative, the processes may be implemented with application specific integrated circuits (ASICs), programmable gate arrays, such as field programmable gate arrays (FPGAs), Programmable Logic Unit (PLU) or Programmable Array Logic (PAL) or any other device capable of executing and responding to other instructions, any other device that may be configured to process electronic signals, or a combination of the devices. The processing device may execute an operating system (OS) and one or more software applications running on the operating system. Further, the processing device may access, store, manipulate, process and generate data in response to the execution of the software. Although. for convenience of understanding, it is sometimes described that one processing device is used, one of ordinary skill in the art may understand that the processing device may include a plurality of processing elements and/or a plurality of types of processing elements. For example, the processing device may include a plurality of processors or one processor and one controller. Further, other processing configurations such as a parallel processor are possible.

Software may include a computer program, code, instruction, or a combination of one or more thereof, and configure the processing device to operate in a desired manner, or instruct the processing devices independently or collectively. Software and/or data may be permanently or temporarily embodied in any type of machine, component, physical device, virtual equipment, computer storage medium or device or in a transmitted signal wave so to be interpreted by the processing device or to provide instructions or data to the processing device. The software may be distributed over networked computer systems and stored or executed in a distributed manner. Software and data may be stored on one or more machine-readable recording media.

Moreover, the objects of the technical solution of the present disclosure or the parts that contribute to the prior art may be implemented in the form of program instructions that may be executed through various computer components and recorded in a machine-readable medium. The machine-readable medium may contain program instructions, data files, data structures, or the like alone or in combination. The program instructions recorded on the machine-readable recording medium may be specially designed and configured for the embodiment, or may be known by a person skilled in the computer software field. Examples of machine-readable recording media include magnetic media such as hard disks, floppy disks and magnetic tapes, optical media such as CD-ROMs, DVDs, and Blu-rays, and floptical disks, and hardware devices specially configured to store and execute program instructions such as ROM, RAM, flash memory, and the like. Examples of program instructions may be composed using a structured programming language such as C, an object-oriented programming language such as C++, or a high-level or low-level programming language (assembly, hardware description languages, and database programming languages and technologies) which may be stored and compiled or interpreted to be executed on any one of the aforementioned devices, as well as a processor, processor architecture, or different hardware and software combinations, or a machine capable of executing any other program instructions. The program instructions includes not only machine code and byte code, but also high-level language code that may be executed by a computer using an interpreter.

Accordingly, in an aspect according to the present disclosure, when the above-described methods and combinations thereof are performed by one or more computing devices, the methods and the combinations of the methods may be implemented using executable codes that perform each of the steps. In another aspect, the method may be implemented using systems that perform the steps, and the methods may be distributed in various ways across devices or all functions may be integrated into one dedicated, standalone device or other hardware. In yet another aspect, the means for performing the steps associated with the processes as described above may include any hardware and/or software as described above. All such sequential combinations are intended to fall within the scope of the present disclosure.

For example, the above described hardware device may be configured to operate as one or more software modules to perform the operation of the embodiment, and vice versa. The hardware device may include a processor such as an MPU, CPU, GPU, or TPU coupled with a memory such as ROM/RAM for storing program instructions and configured to execute the instructions stored in the memory. The hardware device may include a communication unit that may send and receive the signal to and from the external device. In addition, the hardware device may include a keyboard, mouse, or other external input device for receiving instructions written by developers.

In the above, the present disclosure has been described based on details such as specific components and limited embodiments and drawings, which are provided only to help a more general understanding of the present disclosure. The present disclosure is limited to the embodiments. The person with ordinary knowledge in the technical field to which the present disclosure belongs may make various modifications and variations from these descriptions.

Therefore, the idea of the present disclosure is not limited to the described embodiments. Not only the claims attached to the present disclosure, but also all modifications that are equivalent to the claims belong to the scope of the present disclosure. For example, the described steps are performed in a different order from those of the described method, and/or components such as a system, structure, device, circuit, etc. as described are combined with each other in a form different from that of the described method, or other components or are replaced or substituted with an equivalent component, such that an appropriate result may be achieved.

Such equivalent modifications may include, for example, a method that is logically equivalent, capable of producing the same result as that of carrying out the method according to the present disclosure. The true meaning and scope of the present disclosure should not be limited by the above examples, but should be understood in the broadest sense allowed by law.

1-10. (canceled)

11. A method used by a computing device operating based on a machine learning method, the method comprising:
acquiring integrated data of the subject, wherein the integrated data is converted from non-numerical patient data of the subject and numerical patient data of the subject;
applying the integrated data to the machine learning model for criticality assessment of the subject to generate a classification result associated with a criticality of a subject; and
providing information for providing the generated classification result to an external entity,
wherein the integrated data for the non-numerical patient data is expressed as a first vector of a size N (>1), and each element of the first vector corresponds to a respective one of N first categories related to the non-numerical patient data, and wherein an element of the first vector, corresponding to a first category to which the non-numerical patient data belongs, is set to a positive value, and the other element(s) of the first vector is set to zero,
wherein the integrated data for the numerical patient data is expressed as a second vector of a size M (>1), and each element of the second vector corresponds to a respective one of M second categories related to the numerical patient data, and wherein an element of the second vector, corresponding to a second category to which the numerical patient data belongs, is set to a positive value, and the other element(s) of the second vector is set to zero, and
wherein the size M is set to be same as the size N.

12. The method of claim 11, wherein the method further comprises: updating the machine learning model, based on feedback information related to the classification result.

13. The method of claim 11, wherein the criticality of the subject is classified into at least four classes.

14. The method of claim 11, wherein the machine learning model for criticality assessment of the subject is trained using fake symptom occurrence data, and normal symptom data.

15. The method of claim 14, wherein the machine learning model is trained via:
(i) learning the fake symptom occurrence data; and
(ii) after termination of the (i), simultaneously learning the fake symptom occurrence data and the normal symptom data.

16. A computer program comprising instructions stored on a medium, wherein the instructions are implemented to cause a computing device to perform the method of claim 11.

17. The computer program of claim 16, wherein the method further comprises:
updating the machine learning model, based on feedback information related to the classification result.

18. The computer program of claim 16, wherein the criticality of the subject is classified into at least four classes.

19. The computer program of claim 16, wherein the machine learning model for criticality assessment of the subject is trained using fake symptom occurrence data, and normal symptom data.

20. The computer program of claim 19, wherein the machine learning model is trained via:
(i) learning the fake symptom occurrence data; and
(ii) after termination of the (i), simultaneously learning the fake symptom occurrence data and the normal symptom data.

21. A computing device configured to operate based on a machine learning method, the computing device comprising:
a communication unit; and
a processor configured to control the communication unit and to perform:
acquiring integrated data of the subject, wherein the integrated data is converted from non-numerical patient data of the subject and numerical patient data of the subject;
applying the integrated data to the machine learning model for criticality assessment of the subject to generate a classification result associated with a criticality of a subject; and
generating for providing the generated classification result to an external entity,
wherein the integrated data for the non-numerical patient data is expressed as a first vector of a size N (>1), and each element of the first vector corresponds to a respective one of N first categories related to the non-numerical patient data, and wherein an element of the first vector, corresponding to a first category to which the non-numerical patient data belongs, is set to a positive value, and the other element(s) of the first vector is set to zero,
wherein the integrated data for the numerical patient data is expressed as a second vector of a size M (>1), and each element of the second vector corresponds to a respective one of M second categories related to the numerical patient data, and wherein an element of the second vector, corresponding to a second category to which the numerical patient data belongs, is set to a positive value, and the other element(s) of the second vector is set to zero, and
wherein the size M is set to be same as the size N.

22. The computing device of claim 21, wherein the method further comprises (d) updating the machine learning model, based on feedback information related to the classification result.

23. The computing device of claim 21, wherein the criticality of the subject is classified into at least four classes.

24. The computing device of claim 21, wherein the machine learning model for criticality assessment of the subject is trained using fake symptom occurrence data, and normal symptom data.

25. The computing device of claim 24, wherein the machine learning model is trained via:
(i) learning the fake symptom occurrence data; and
(ii) after termination of the (i), simultaneously learning the fake symptom occurrence data and the normal symptom data.
